(19) United States
(12) Patent Application Publication
(10) Pub. No.: US 2016/0166485 A1
(43) Pub. Date: Jun. 16, 2016

(54) Transparent Sunscreen Compositions and Use Thereof

(71) Applicant: SASOL GERMANY GMBH, Hamburg (DE)

(72) Inventors: Michael BROCK, Schermbeck (DE); Petra LEWING, Heiden (DE)

(21) Appl. No.: 14/904,808

(22) PCT Filed: Jul. 23, 2014
(86) PCT No.: PCT/DE2014/000371

§ 371 Date: Jan. 13, 2016

(30) Foreign Application Priority Data
Jul. 26, 2013 (DE) 20 2013 103 395.2

(51) Int. Cl. A61K 8/37

(52) U.S. Cl. CPC A61K 8/37; A61Q 17/04

(57) ABSTRACT

The invention relates to transparent sunscreen compositions containing an ester of a α-hydroxy carboxylic acid and a solid organic UV-Filter, which is present in the dissolved state, and which are essentially free of water and ethanol, and to the use thereof as a sunscreen, in particular sun protection gel or sun protection spray.

The invention relates to transparent sunscreen compositions containing at least one ester of an α-hydroxycarboxylic acid and a solid organic UV filter, which is present in the dissolved state, and which are essentially free of water and ethanol, and to the use thereof as a sunscreen, in particular sun protection gel or sun protection spray.

### PRIOR ART

For years, there has been an unbroken trend to favor suntanned skin. To achieve this, people expose their skin to solar radiation. Alternatively, they can visit tanning salons. However, The UVA and UVB radiation of sunlight has a damaging effect on the skin. When the human skin is exposed to the sun for a longer duration, this can lead to various disorders. For example, mention is made of erythema, dermatosis, skin infections, accelerated aging of the skin, and skin cancer. That is why numerous sunscreens for protecting the skin are commercially available. Among other components, they contain light-protection filters that can be used in cosmetic preparations. In most states, these UVA and UVB filters are compiled in the form of positive lists such as Appendix IV of the European Cosmetic Agents Regulation No. 1223/2009.

The large number of commercially available sunscreens should not detract from the fact that these preparations of the prior art have a number of disadvantages. Thus, many sunscreens are creams or lotions that are O/W or W/O emulsions, in contrast to the present invention. Experience has shown that consumers of this type of sunscreens often apply too little to the skin, so that the sunscreen factor indicated on the packaging is not reached. Moreover, in addition to organic UV filters, many of the sunscreens known from the prior art contain inorganic UV filters such as TiO₂ or ZnO, which are present in the form of small solid particles, and accordingly they are present as dispersions and therefore do not lead to transparent sunscreen compositions, and, when used on the skin, they often form a white deposit which the consumer considers unsightly.

Since, in contrast to the present invention, the mentioned sunscreens represent emulsions as well as dispersions, they contain emulsifiers and dispersants. In the case of high humidity due to sweating or bathing, for example, the emulsifiers and dispersants can re-emulsify considerable proportions of the UVA and UVB filters, after the application of the sunscreen to the skin, and remove them from the skin, so that they are no longer available for protection from the sun.

Alternatively, DE 20200006005 U1 and DE 102005059742 A1 propose transparent alcohol-based sun protection gels or sprays, which are used increasingly by many consumers due to their appearance which is associated with freshness, transparency and naturalness. These transparent sun protection gels or sprays contain at least 20 wt % of ethanol. The presence of ethanol is mentioned in advertising because of its refreshing-cooling sensory effect. However, it is known that ethanol can dry and irritate the skin. In addition, the use of ethanol-containing sunscreens is not suitable for certain consumer groups such as babies and infants, for example.

Alternatively, DE 602004009901 T2 claims, in particular, transparent and gel-like sunscreens that contain carriers. The carriers represent oils such as, for example, isopropyl myristate, isopropyl palmitate, alkylbenzoate, preferably mineral oil. The sole purpose of these oils is to dissolve the organic UV filters. They have no moisture-releasing or skin-calming effect, which would be very advantageous for skin exposed to the sun. The mineral oil is also thought to function as a softening agent. On the other hand, it is known that mineral oils close the skin pores and as a result minimize skin respiration.

EP 0685226 B1 claims cosmetic compositions containing, among other components, the solid organic UV filter ethylhexyl triazone in combination with esters of α-hydroxycarboxylic acids. There are no indications that the claimed cosmetic compositions are transparent; on the contrary, they are water-containing emulsions, to the extent that they are sunscreen formulations.

EP 0904776 B1 describes a composition consisting of at least one UV filter of the filter type derived from benzylidene camphor, dibenzoylmethane and/or triazine, in mixtures with a dialkyl tartrate. Here too, there is no indication that the claimed composition is suitable for producing transparent sunscreens.

EP 0800816 B1 claims active substance combinations with light-protection action consisting of a triazine derivative as UV filter and one or more dialkyl esters of α,β-dihydroxycarboxylic acids. There is no indication that the claimed compositions are suitable for producing transparent sunscreens; on the contrary, they are water-containing emulsions.

### OBJECT OF THE INVENTION

The object of the present invention consisted in providing a remedy for the mentioned disadvantages. Surprisingly, it was found that the sunscreen compositions, or their use according to the claims, procure a remedy for the mentioned disadvantages.

### SUMMARY OF THE INVENTION

The present invention relates to transparent sunscreen compositions containing esters of α-hydroxycarboxylic acids, in a further embodiment, the present invention relates to the use of sunscreen compositions as sunscreen sprays that can be sprayed on the skin and as gels that can be applied by hand to the skin. Due to the fact that the sunscreen compositions are transparent liquids, they are not in the form of emulsions and/or dispersions. There is no need to use surfactants, and the transparent sunscreen compositions are therefore preferably surfactant-free.

In detail, the present invention relates to transparent sunscreen compositions containing
(a) at least one ester of an α-hydroxycarboxylic acid and thereof preferably 5 to 95 wt %, particularly preferably 10 to 85 wt %, and more particularly preferably 15 to 80 wt %:
(b) at least one water-insoluble and solid organic UV filter, in particular, a UVA and a UVB
(c) at most 1 wt % of water; and
(d) at most 1 wt % of ethanol;
   and optionally
(e) 0-10 wt % of one or more thickeners, comprising hydrophilic amorphous silicates, hydrophobic amorphous silicates, silicate salts or their mixtures:
   and also, independently of (e), possibly optionally, in particular
(f) 0-60 wt %, preferentially 5 to 50 wt %, in particular preferably 10 preferably 40 wt % of saturated or unsaturated, branched or linear alcohols having a chain length of 10 to 30 C atoms, preferably C10 to C30 Guerbet alcohols or their mixtures.

The sunscreen compositions according to the invention are considered to be transparent, if it is possible, in daylight, to see with the naked eye through a disposable cuvette (company Brand GmbH, Wertheim/Germany, dimensions: 12.5×12.5×45 mm, wavelength range: 300 nm-900 nm) filled with the sunscreen composition according to the invention. Letters (font type: Arial, size: 8) that are located immediately behind the disposable cuvette have to be clearly recognizable and readable. “Insoluble in water” or “water-insoluble” means that less than 0.5 wt % of the organic UV filter dissolves in water at 25° C. “Solid” means that the organic UV filter is a solid at 25° C., in particular that it is present in crystalline form.

### DETAILED DESCRIPTION OF THE INVENTION

α-Hydroxycarboxylic acids such as, for example, lactic acid, citric acid, malic acid or tartaric acid, are used extensively in cosmetic preparations. They are used as moisturizer and for exciting the cell metabolism in the skin in case of stressed and aging skin. Since α-hydroxycarboxylic acids can lead to skin irritation already at low concentrations, their use in cosmetic preparations is strongly limited. A remedy is provided by esters of α-hydroxycarboxylic acids, which can be considered to be carriers of α-hydroxycarboxylic acids and which are cleaved slowly by the esterases present on and in the skin, releasing the α-hydroxycarboxylic acids.

This cleavage occurs slowly, so that esters of α-hydroxycarboxylic acid exert a long-term treatment effect on the skin. Due to the slow release of the α-hydroxycarboxylic acids on the skin, it is possible, in comparison to free o-hydroxycarboxylic acids, to apply large amounts of the esters of the α-hydroxycarboxylic acids by means of cosmetic preparations on the skin, where they have a moisture-releasing and calming effect. All of the acid groups of the α-hydroxycarboxylic acid(s) used according to the invention are therefore esterified,

The transparent sunscreen compositions according to the invention contain at least one ester of an α-hydroxycarboxylic acid as acid component, and R—OH as alcohol component, where R═C₈-C₂₀ alkyl, saturated or unsaturated, linear or branched, or their mixtures. It is particularly preferable if the esters are esters of lactic acid, citric acid, malic acid, tartaric acid or their mixtures as acid component, and R—O(H) as alcohol component, where R═C₈-C₂₀ alkyl, saturated or unsaturated, linear or branched, or their mixtures.

Such esters of α-hydroxycarboxylic acids are commercially available from Sasol/Italy under the names COSMACOL® ELI (INCI: C12-13 Alkyl Lactate), COSMACOL® ECI (INCI: Tri-C12-13 Alkyl Citrate), COSMACOL® ECL (INCI: Tri-C14-15 Alkyl Citrate), COSMACOL® EMI (INCI: Di-C12-13 Alkyl Malate), COSMACOL® ETI (INCI: Di-C12-13 Alkyl Tartrate) as well as COSMACOL® ETLP (INCI: Dimyristyl Tartrate). In addition to having a moisture-releasing and calming effect on the sun stressed skin, these esters are characterized in that they represent good solvents for organic UV filters, particularly solid organic UV filters.

Moreover, the sunscreen compositions according to the invention contain at least one water-insoluble and solid organic UV and/or UVB filter as mentioned in Appendix VI of the European Cosmetic Agents Regulation No. 1223/2009. Particularly preferable are diethylamino hydroxybenzoyl hexyl benzoate (Uvinul® A Plus, BASF), ethylhexyl methoxycinnamate (for example, Eusole® 2292, Merck), ethyl triazone (Uvinul® T 150, BASF), bis-ethylhexyloxyphenol methoxyphenyl triazine (Tinosorb® S. BASF) as well as butyl methoxydibenzoylmethane (Eusolex® 9020, Merck).

The water-insoluble and solid organic UV filters are soluble in the esters of the α-hydroxycarboxylic acids or their mixtures and in the sunscreen compositions. Therefore, the sunscreen compositions are also transparent. In addition to the at least one water-insoluble and solid organic UVA or UVB filter or their mixtures, liquid soluble UVA or UVB filters or their mixtures, for example, can also be present.

The transparent sunscreen composition according to the invention contain at most 1 wt % of water and at most 1 wt % of ethanol and are thus substantially free of water and substantially free of ethanol. Moreover, the transparent sunscreen compositions according to the invention preferentially contain at most 1 wt % of a C3 alcohol and preferably at most 1 wt % of a C4 alcohol.

In an additional embodiment of the present invention, as a result of the use of one or more thickeners comprising hydrophilic amorphous silicates, hydrophobic amorphous silicates, silicate salts or their mixtures, transparent and gel-like sunscreen compositions can be produced. Examples are the Aerosil® and Sipernat® types from Evonik Industries and the Wacker® HDK types from Wacker Chemie.

When used as sprays, the transparent sunscreen compositions according to the invention contain 0 to 2 wt %, in particular 0 to 1 wt %, such as 0.01 to 0.1 wt %, of the above thickeners. When used as gels, the transparent sunscreen compositions according to the invention contain 0-10 wt %, in particular 1 to 8 wt % and particularly preferably 1.5 to 4 wt % of the above thickeners.

Moreover, in addition to the esters of the α-hydroxycarboxylic acids, the transparent sunscreen compositions according to the invention can contain additional oils or waxes or their mixtures. Preferentially, the oils and/or waxes are selected from the group of the lecithins, triglycerides of saturated or unsaturated, branched and/or linear alkylcarboxylic acids having a chain length of 6 to 24, in particular 12 to 18 C atoms, including their mixtures.

The triglycerides preferentially can be synthetic, semisynthetic or natural oils and/or waxes such as, for example, triisostearin, soybean oil, castor-bean oil, olive oil, safflower oil, wheat germ oil, grape seed oil, peanut oil, sunflower oil, almond oil, palm oil, palm kernel oil, coconut oil, thistle oil, evening primrose oil, rapeseed oil and the like.

Moreover, the oils and/or waxes can consist of the group of the branched and/or linear hydrocarbons such as, for example, paraffin waxes. Vaseline (petrolatum), mineral oil, paraffin oil, isohexadecane, dodecane, tetradecane, hexadecane, octadecane, docosane and polyolefins. According to the present invention, the oils and/or waxes can also be selected preferentially from the group of the esters of saturated and/or unsaturated, branched and/or linear alkylcarboxylic acids having a chain length of 3 to 30 C atoms, and of saturated or unsaturated, branched or linear alcohols having a chain length of 3 to 30 C atoms, including their mixtures. As examples are mentioned: jojoba oil, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate and erucyl erucate. Furthermore, the emulsions according to the invention can contain oils and/or waxes from the groups of the silicone oils (cyclic and/or linear) and the group of the dialkyl ethers such as, for example, dicaprylyl ether or distearyl ether, or the group of the dialkyl carbonates. Esters of aromatic carboxylic acids with saturated or unsaturated, branched or linear alcohols having a chain length of 3 to 30 C atoms, including their mixtures such as C₁₂₋₁₅ alkyl benzoate, for example, can also be used in the transparent sunscreens according to the invention. In addition, any mixtures of such oil and wax components can be used preferentially in the sense of the present invention.

The transparent sunscreen compositions according to the invention preferably contain saturated or unsaturated, branched or linear alcohols having a chain length of 10 to 30 C atoms, including their mixtures. Guerbet alcohols are particularly preferable, such as ISOFOL® 12 (INCI: Butyloctanol, Sasol Germany), ISOFOL® 16 (INCI: Hexyldecanol), ISOFOL® 18 T (INCI: Octyldecanol) or ISOFOL® 20 (INCI: Octyldodecanol), including their mixtures.

Moreover, the transparent sunscreen compositions according to the invention can contain other cosmetic and/or pharmaceutical excipients, additives and/or active substances. They are, for example: cooling substances such as, for example, menthol, dyes, perfume, antioxidants, plant extracts, antiperspiration agents, tanning agents, film formers, protein hydrolysates, vitamins, antimicrobial substances and the like.

### Examples

The following examples are intended to illustrate the invention without limiting it. All the quantity indications are based on the weight and the total quantity of the preparations.

Phase A was heated at approximately 60° C. and stirred until the sample turned transparent. The sample was allowed to cool to approximately 30° C., and phase B was added and homogenized. For the production of the gels, phase C was added and homogenized using an Ultra-Turrax. Subsequently, the gel was defoamed under a vacuum.

Determination of the clear melt point: A test tube or Shukoff flask was filled with 5-10 of the clear product to be tested and provided with a low-temperature thermometer. The sample was cooled in the cryostat bath to at least −20° C., until the sample was completely frozen. Subsequently, the sample was slowly thawed at room temperature. The temperature at which the sample no longer displays any detectable turbidity was considered the clear melting point. As the result, the arithmetic mean from two determinations was indicated, provided their difference did not exceed 1° C.

The respective formulations and results of the investigations are represented in Tables 1a, 1 b, 2a and 2b.

TABLE 1a
Transparent sunscreen sprays - formulations:

| | | | | Preparation | | | |
|---|---|---|---|---|---|---|---|
| | | | | [parts by weight] | | | |
| Phase | Product name | *) | INCI name | 1 | 2 | 3 | 4 |
| A | Uvinul A Plus | 3 | 2-(4-Ethylamino-2-hydroxybenzoyl)-benzoic acid | 10.0 | 10.0 | / | 10.0 |
| | | | hexyl ester | | | | |
| | Eusolex 2292 | 4 | Ethylhexyl methoxycinnamate | 8.0 | 8.0 | / | 10.0 |
| | Uvinul T150 | 3 | Ethylhexyl triazone | 2.0 | 2.0 | / | 5.0 |
| | Eusolex OCR | 4 | Octocrylene | / | / | 10.0 | / |
| | Eusolex HMS | 4 | Homosalate | / | / | 10.0 | / |
| | Eusolex 9020 | 4 | Butyl methoxydibenzoylmethane | / | / | 5.0 | / |
| | Tinosorb S | 3 | Bis-ethylhexyloxyphenol methoxyphenyl triazine | / | / | 3.5 | / |
| | Lexfilm Sun | 5 | Polyester-7 (and) neopentyl glycol diheptanoate | 1.0 | 1.0 | 1.0 | 1.0 |
| | COSMACOL EBI | 2 | C12-15 alkyl benzoate | / | 30.0 | / | 20.0 |
| | COSMACOL ELI | 2 | C12-13 alkyl lactate | 75.0 | 5.0 | 46.5 | 20.0 |
| | COSMACOL EMI | 2 | Di C12-13 alkyl malate | / | 5.0 | 10.0 | 5.0 |
| | COSMACOL ETI | 2 | Di C12-13 alkyl tartrate | / | 5.0 | 10.0 | 5.0 |
| B | ISOFOL 16 | 1 | Hexyldecanol | / | / | / | 20.0 |
| | ISOFOL 20 | 1 | Octyldodecanol | / | 30.0 | / | / |
| | D-Panthenol (99%) | 6 | Panthenol | 0.75 | 0.75 | 0.75 | 0.75 |
| | Tocopherol | 4 | Tocopherol | 0.25 | 0.25 | 0.25 | 0.25 |

TABLE 1b
Transparent sunscreen sprays - properties:

| | Preparation | | | |
|---|---|---|---|---|
| | 1 | 2 | 3 | 4 |
| Appearance after | trans- | trans- | trans- | trans- |
| 4 weeks at room | parent | parent | parent | parent |
| temperature | | | | |
| Appearance after | trans- | trans- | trans- | trans- |
| 4 weeks at −10° C. | parent | parent | parent | parent |
| Information SPF - | 30 | 30 | 30 | 50 |
| according to BASF | | | | |
| Sunscreen Simulator ¹) | | | | |
| calculated SPF | 35.5 | 35.5 | 35.9 | 51.4 |
| UVA PF/SPF | >0.33 | >0.33 | >0.33 | >0.33 |
| critical wavelength (nm) | 379 | 379 | 378 | 377 |
| Asterisks (evaluation) | *** | *** | **** | *** |
| Clear melting point | <−20° C. | <−20° C. | <−20° C. | <−20° C. |

TABLE 2a
Transparent sun protection gels - formulations:

| | | | | Preparation | | | |
|---|---|---|---|---|---|---|---|
| | | | | [parts by weight] | | | |
| Phase | Product name | *) | INCI name | 5 | 6 | 7 | 8 |
| A | Uvinul A Plus | 3 | 2-(4-Ethylamino-2-hydroxybenzoyl)-benzoic acid | 10.0 | 10.0 | 10.0 | / |
| | | | hexyl ester | | | | |
| | Eusolex 2292 | 4 | Ethylhexyl methoxycinnamate | 8.0 | 8.0 | 8.0 | / |
| | Uvinul T150 | 3 | Ethylhexyl triazone | 2.0 | 2.0 | 2.0 | / |
| | Eusolex OCR | 4 | Octocrylene | / | / | / | 10.0 |
| | Eusolex HMS | 4 | Homosalate | / | / | / | 10.0 |
| | Eusolex 9020 | 4 | Butyl methoxydibenzoylmethane | / | / | / | 5.0 |
| | Tinosorb S | 3 | Bis-ethylhexyloxyphenol methoxyphenyl triazine | / | / | / | 3.5 |
| | Lexfilm Sun | 5 | Polyester-7 (and) neopentyl glycol diheptanoate | 1.0 | 1.0 | 1.0 | 1.0 |
| | COSMACOL EBI | 2 | C12-15 alkyl benzoate | / | 20.0 | / | / |
| | COSMACOL ELI | 2 | C12-13 alkyl lactate | / | / | 15.0 | 31.5 |
| | COSMACOL ECI | 2 | Tri C12-13 alkyl citrate | / | 14.0 | / | / |
| | COSMACOL EMI | 2 | Di C12-13 alkyl malate | 75.0 | 5.0 | 15.0 | 5.0 |
| | COSMACOL ETI | 2 | Di C12-13 alkyl tartrate | / | / | 15.0 | 5.0 |
| B | ISOFOL 16 | 1 | Hexyldecanol | / | / | 30.0 | / |
| | ISOFOL 18 T | 1 | Octyldecanol | / | / | / | 25.0 |
| | ISOFOL 20 | 1 | Octyldodecanol | / | 36.0 | / | / |
| | D-Panthenol (99%) | 6 | Panthenol | 0.75 | 0.75 | 0.75 | 0.75 |
| | Tocopherol | 4 | Tocopherol | 0.25 | 0.25 | 0.25 | 0.25 |
| C | Aerosil 200 | 7 | Silica | / | 3.0 | / | / |
| | Aerosil 200V | 7 | Silica | 3.0 | / | 3.0 | 3.0 |

TABLE 2b
Transparent sun protection gels - properties:

| | Preparation | | | |
|---|---|---|---|---|
| | 5 | 6 | 7 | 8 |
| Appearance after | trans- | trans- | trans- | trans- |
| 4 weeks at room | parent | parent | parent | parent |
| temperature | | | | |
| Appearance after | trans- | trans- | trans- | trans- |
| 4 weeks at 40° C. | parent | parent | parent | parent |
| Appearance after | trans- | trans- | trans- | trans- |
| 4 weeks at −10° C. | parent | parent | parent | parent |
| Information SPF - | 30 | 30 | 30 | 30 |
| according to BASF | | | | |
| Sunscreen Simulator ¹) | | | | |
| calculated SPF | 35.5 | 35.5 | 35.5 | 35.9 |
| UVA_PF/SPF | >0.33 | >0.33 | >0.33 | >0.33 |
| critical wavelength (nm) | 379 | 379 | 379 | 378 |
| Asterisks (evaluation) | *** | *** | *** | **** |
| Clear melting point | <−20° C. | <−20° C. | <−20° C. | <−20° C. |
| Viscosity in mPas | 10500 | 39000 | 7200 | 5000 |
| (25° C.; 1 1/s; | | | | |
| Haake RS1; C35/2°) | | | | |

### Legend for Tables 1a and 2a

*) Manufacturer:

(1)=Sasol Germany GmbH

(2)=Sasol Italy S.p.A.

(3)=BASF AG

(4)=Merck KgaA

(5)=Innolex Chemical Co.

(6)=DSM Nutritional Products Ltd.

(7)=Evonik Ind, AG

1) www.sunscreensimulaor.basf.com/Sunscreen_Simulator/Login_show.action

1. Transparent sunscreen composition, which is a transparent liquid and can be sprayed by means of a hand pump as a sunscreen spray on the skin, containing
(a) at least one ester of an α-hydroxycarboxylic acid,
(b) at least one water-insoluble and solid organic UV filter,
(c) at most 1 wt % of water, and
(d) at most 1 wt % of ethanol.

2. Sunscreen composition according to claim 1, further comprising:
(f) 0 to 60 wt % of saturated or unsaturated, branched or linear alcohols having a chain length of 10 to 30 C atoms or their mixtures.

3. Sunscreen composition according to claim 1, wherein the ester of an α-hydroxycarboxylic acid comprises, as alcohol component, R—O—(H) alcohols with R C8-C20 alkyl, saturated or unsaturated, linear or branched or their mixtures.

4. Sunscreen composition according to claim 1, wherein the ester of an α-hydroxycarboxylic acid comprises (a), as acid component, lactic acid, citric acid, malic acid, tartaric acid or their mixtures.

5. Sunscreen composition according to claim 1, wherein 5 to 95 wt % of the esters of an α-hydroxycarboxylic acid are contained in the sunscreen composition.

6. Sunscreen composition according to claim 1, wherein the water-insoluble and solid organic UV filters are or comprise diethylamino hydroxybenzoyl hexyl benzoate, ethylhexul methoxycinnamate, ethyl triazone, bis-ethylhexyloxyphenol methoxypheyl triazine, butyl methoxydibenzoylmethane or their mixtures.

7. Sunscreen composition according to claim 1, further comprising:
(g) oils or waxes or their mixtures.

8. Sunscreen composition according to claim 1, wherein the sunscreen composition contains 0 to 2 wt % thickener.

9-10. (canceled)

11. Sunscreen composition according to claim 2, wherein said alcohols are C10 to C30 Guerbet alcohols.

12. Sunscreen composition according to claim 1, further comprising:
(f) 5 to 50 wt % of saturated or unsaturated, branched or linear alcohols having a chain length of 10 to 30 C atoms or their mixtures.

13. Sunscreen composition according to claim 12, wherein said alcohols are C10 to C30 Guerbet alcohols.

14. Sunscreen composition according to claim 1, further comprising:
(f) 10 to 40 wt % of saturated or unsaturated, branched or linear alcohols having a chain length of 10 to 30 C atoms or their mixtures.

15. Sunscreen composition according to claim 14, wherein said alcohols are C10 to C30 Guerbet alcohols.

16. Sunscreen composition according to claim 1, wherein 10 to 85 wt % of the esters of an α-hydroxycarboxylic acid are contained in the sunscreen composition.

17. Sunscreen composition according to claim 1, wherein 15 to 80 wt % of the esters of an α-hydroxycarboxylic acid are contained in the sunscreen composition.

18. A process of using the sunscreen composition of claim 1, comprising:
providing the sunscreen composition of claim 1, said sunscreen composition being sprayable with a hand pump; and
applying said sunscreen composition on skin.

19. The process of claim 18, wherein the sunscreen composition contains 0 to 2 wt % thickener.
